(19) United States
(12) Patent Application Publication
(10) Pub. No.: US 2020/0337436 A1
(43) Pub. Date: Oct. 29, 2020

(54) PROCESS FOR APPLICATION OF LIQUID COSMETIC MATERIAL AND APPARATUS

(71) Applicant: Nicole Druckman, Massapequa, NY (US)

(72) Inventor: Nicole Druckman, Massapequa, NY (US)

(21) Appl. No.: 16/856,991

(22) Filed: Apr. 23, 2020

Related U.S. Application Data
(60) Provisional application No. 62/838,099, filed on Apr. 24, 2019.

(51) Int. Cl. A45D 34/04; A61K 8/02; A61Q 1/12

(52) U.S. Cl. CPC A45D 34/042; A45D 2034/007; A61Q 1/12; A61K 8/022

(57) ABSTRACT

Liquid cosmetic material can be applied by first stirring the material on a silicone surface to improve flow.

### CROSS-REFERENCE TO RELATED APPLICATION

The present application claims the priority of U.S. Provisional Patent Application 62/838,099, filed Apr. 24, 2019, which is incorporated herein by reference.

### BACKGROUND OF THE INVENTION

### 1. Field of the Invention

The present invention relates to processes for the application of cosmetics materials and related apparatus.

### 2. Brief Description of the Prior Art

The application of cosmetic materials to human skin has its origins in prehistory. Many different types of materials have been used over the ages to enhance the appearance. Similarly, a variety of application techniques have been used, largely dependent on the physical characteristics of the cosmetic material. In modern times, the application of cosmetics, or “make-up,” has become a sophisticated artform, frequently involving cosmetics intended for use on specific portions of the body or to provide a special effect. Beginning in the early part of the Twentieth Century, the concept of using a base of “foundation” or other cosmetic material was introduced for actors in the theater and motion pictures. This was an attempt to mimic the way in which artists seek to begin with a fresh, uniform canvas, by using first a gesso base coat. Cosmetic foundations now are available in a variety of physical states, including liquid, cream, cream-to-powder, stick, powder, mineral, and tinted moisturizer. Whatever the physical form, cosmetic foundations typically include various moisturizers, colorants and fillers, with a base selected from water, an oil, or wax. Talc is a common ingredient to promote smooth application of the foundation, while colorants such as titanium dioxide, and other metal oxides are selected to provide a natural skin tone. Foundations may also include materials selected to address specific skin issues, as overly dry or oily skin. A foundation may also include light-scattering materials to provide a more even appearance. Similarly, a foundation may also include material to hydrate the skin to help mask small imperfections such a wrinkles. A continuing problem associated with some foundation materials is that they tend to lose moisture over time, and/or “set” by becoming less fluid as components of the foundation interact over time. Foundations, like other cosmetic materials, can be very expensive, so that foundation left over after an application which can subsequently dry out or otherwise become unusable, can be a significant loss.

### SUMMARY OF THE INVENTION

The present invention provides a process for application of a liquid cosmetic material to human skin, the process includes providing a long-term storage container for the cosmetic material; providing a storage compartment for the cosmetic material in the storage container; providing a pre-application area in the storage container; providing a silicone surface on the pre-application area; and providing at least one cosmetic material in the storage compartment. Preferably, the process further includes providing a cosmetic application tool; applying the at least one cosmetic material to the cosmetic application tool; contacting the silicone surface with the cosmetic application tool bearing the cosmetic material; moving the cosmetic application tool over the silicone surface with the application of pressure against the silicone surface to increase the temperature of the at least one cosmetic material; and subsequently applying the at least one cosmetic material to human skin. Preferably, the at least one cosmetic material is a foundation material. In one presently preferred embodiment, the at least one cosmetic material comprises at least two cosmetic materials, and further comprises mixing the at least two cosmetic materials together with the cosmetic application tool. In this embodiment, the at least two cosmetic materials preferably include at least one foundation material and at least one additive. Preferably, the at least one additive comprises a translucent powder material. Preferably, the foundation material is selected from the group consisting of oil-based foundations, emollient-based foundations, alcohol-based foundation, powder-based foundations, mineral-based foundations, water-based foundations, and silicone-based foundations. Preferably, the cosmetic application tool is selected from the group consisting of sponges, brushes and air-brushes.

In another aspect, the present invention provides a process for application of a liquid cosmetic material to human skin. In this aspect the process includes: providing at least one cosmetic material; providing a silicone surface; providing a cosmetic application tool; applying the cosmetic material to the cosmetic application tool; rubbing the cosmetic application tool bearing the at least one cosmetic material against the silicone surface to generate friction heat to increase the temperature of the cosmetic material; and subsequently applying the heated cosmetic material to human skin. Preferably, the at least one cosmetic material is a foundation material. Preferably, the foundation material is selected from the group consisting of oil-based foundations, emollient-based foundations, alcohol-based foundations, powder based foundations, mineral-based foundations, water-based foundations, and silicone-based foundations. Preferably, the cosmetic application tool is selected from the group consisting of sponges, brushes and air-brushes. In another aspect of the present invention, the at least one cosmetic material comprises at least two cosmetic materials, and further comprises mixing the at least two cosmetic materials together with the cosmetic application tool. In this aspect, the at least two cosmetic materials preferably include at least one foundation material and at least one additive. Preferably, the at least one additive comprises a translucent powder material.

The present invention also provides a storage container for a cosmetic material having an open position and a closed position. The storage container of the present invention includes a first section having a compartment formed therein for containing the cosmetic material, the first section having a first sealing surface, and a second section having a silicone surface for frictionally heating the cosmetic material, the second section having a second sealing surface, wherein the first sealing surface and the second sealing surface cooperate to seal the container in the closed position. Preferably, the silicone surface has a plurality of raised portions. Preferably, the plurality of raised portions are generally circular, and regularly spaced within the second section. Preferably, the raised portions are truncated generally spherical sections in shape. Preferably, the storage container further includes a hinge for connecting the first section and the second section. Preferably, the storage container further includes a lock for securing the storage container in the closed position. Preferably, the first section and the second section are cylindrical or hexagonal.

### BRIEF DESCRIPTION OF THE DRAWINGS

FIG. 1 is a front perspective view of a cosmetics storage container of the present invention shown in an open position to reveal the interior thereof.

FIG. 2 is a rear elevational view of the cosmetics storage container of FIG. 1, shown in the closed position.

FIG. 3 is a rear elevational view of the cosmetics storage container of FIG. 1 shown in an open position.

FIG. 4 is a rear perspective view of the cosmetics storage container of FIG. 1 shown in the closed position.

### DETAILED DESCRIPTION

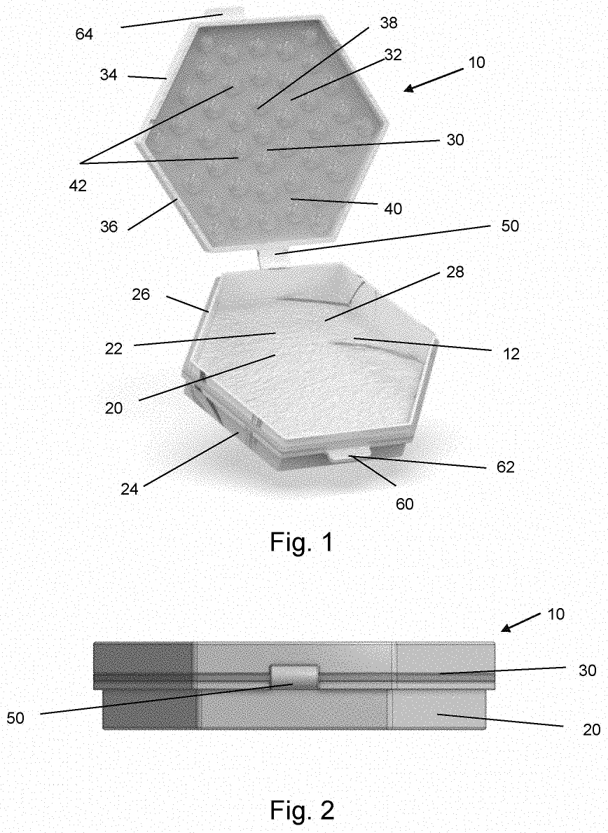

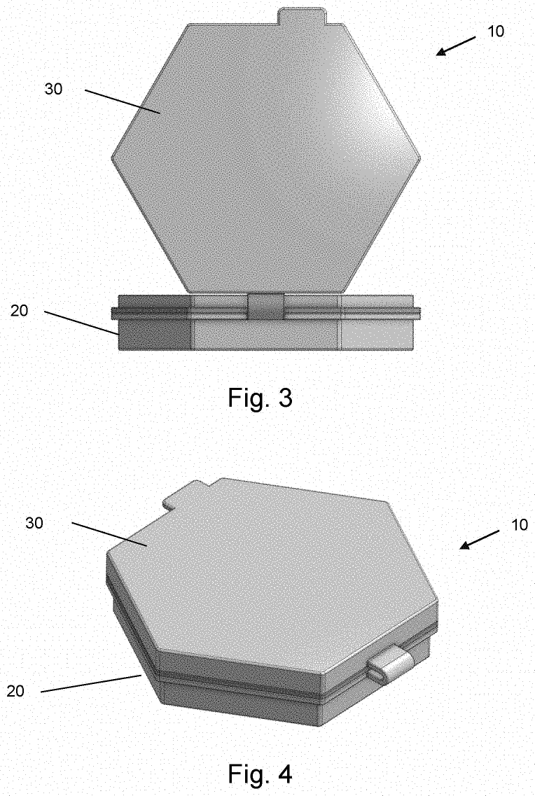

A cosmetics storage container according to the present invention is shown in the Figures, in which like elements are designated by like reference numerals. FIG. 1 provides a front perspective view of a cosmetics storage container 10 of the present invention shown in an open position to reveal the interior 12 thereof. The container 10 includes a first or lower section 20 having a generally hexagonal base 22 enclosed on each side thereof by a low wall 24 thus forming a storage location or compartment 28 for receiving a liquid or semisolid cosmetics material (not shown). At the upper end of the wall 24 of the first section 20 a first sealing surface 26 is provided. The container 10 also includes a second or upper section 30 which also includes a base 32 having a generally hexagonal shape, as well as a low wall 34 extending from each side of the base 32. The upper section 30 provides a pre-application area 38 for the cosmetic material within the storage container 10. At the lower end of the wall 34 of the second section 30 a second sealing surface 36 is provided for sealing engagement with the first sealing surface 26 when the container 10 is closed. The base 32 of the upper section 30 is slightly larger than the base 22 of the lower section 20, as best seen in the rear elevational view of FIG. 2. The interior of the base 32 of the upper section 30 is provided with a textured surface 40, preferably a silicone surface, on which a plurality of raised portions 42 are provided. Preferably, the raised portions 42 take the form of “bumps” or truncated portion of generally spherical surfaces. The raised portions 42 are preferably uniformly distributed over the interior of the base 32 of the second section. In use, a brush or sponge or other application tool (not shown) is used to take up a portion of cosmetic material stored in the first section 20 and stirred on the textured surface 40 of the upper section to liquefy, warm, and/or to otherwise prepare the cosmetic material for application to the skin. The first section 20 and the second section 30 are connected together by a hinge 50 extending from the rear of the first and second sections 20, 30 (best seen in FIGS. 3 and 4). In addition, a lock 60 is provided opposite the hinge 50 on the front of the container 10. A first section or first tab 62 is provided on the first section 20 and a second section of second tab 64 of the lock 60 is provided on the second section 30 such that when closed the container 10 can be readily opened by forcing apart the first tab 62 and the second tab 64.

In another aspect, the present invention provides a cosmetics application kit including a storage container 10 of the present invention and at least one cosmetics application tool, such as a brush or sponge.

The container 10 can be used in the process of the present invention. In that process, which entails the application of a liquid or semi-solid cosmetic material to human skin, a long-term storage container for the cosmetic material is provided. A storage compartment for the cosmetic material is provided for in the storage container. A pre-application area is also provided in the storage container. Preferably, a silicone surface, which is preferably textured, is provided in or on the pre-application area. The surface in the pre-application area can be a surface formed from another material, such as a suitable plastic material, such as an appropriate polyethylene, polytetrafluoroethylene, or like material, having a non-stick surface, thus having a low coefficient of friction. Further the process includes providing at least one cosmetic material in the storage compartment, such as a liquid or semi-solid cosmetic foundation.

In addition, the process can further include providing a cosmetic application tool and applying the at least one cosmetic material to the cosmetic application tool. Preferably, in this aspect the process also includes contacting the silicone surface with the cosmetic application tool bearing the cosmetic material, moving the cosmetic application tool over the silicone surface with the application of pressure against the silicone surface to increase the fluidity or temperature of the at least one cosmetic material, and subsequently applying the at least one cosmetic material to human skin.

The cosmetic material employed in the present process can be a foundation material. In another aspect, at least two cosmetic materials are employed in the process, and the at least two cosmetic materials are mixed together with the cosmetic application tool. For example, the at least two cosmetic materials can include at least one foundation material and at least one additive, such as a translucent powder material. The foundation material employed in the process can be selected from the group consisting of oil-based foundations, emollient-based foundations, alcohol-based foundation, powder-based foundations, mineral-based foundations, water-based foundations, and silicone-based foundations. The cosmetic application tool can be selected from the group consisting of sponges, brushes and air-brushes.

Various modifications can be made in the details of the various embodiments of the articles of the present invention, all within the scope and spirit of the invention as defined by the appended claims.

1. A process for application of a liquid cosmetic material to human skin, the process comprising:
a) providing a long-term storage container for the cosmetic material;
b) providing a storage compartment for the cosmetic material in the storage container;
c) providing a pre-application area in the storage container;
d) providing a silicone surface on the pre-application area; and
e) providing at least one cosmetic material in the storage compartment.

2. A process according to claim 1 further comprising:
a) providing a cosmetic application tool;
b) applying the at least one cosmetic material to the cosmetic application tool;
c) contacting the silicone surface with the cosmetic application tool bearing the cosmetic material;
d) moving the cosmetic application tool over the silicone surface with the application of pressure against the silicone surface to increase the temperature of the at least one cosmetic material; and
e) subsequently applying the at least one cosmetic material to human skin.

3. A process according claim 1 wherein the at least one cosmetic material is a foundation material.

4. A process according to claim 1 wherein the at least one cosmetic material comprises at least two cosmetic materials, and further comprises mixing the at least two cosmetic materials together with the cosmetic application tool.

5. A process according to claim 4, wherein the at least two cosmetic materials include at least one foundation material and at least one additive.

6. A process according to claim 5 wherein the at least one additive comprises a translucent powder material.

7. A process according to claim 3 wherein the foundation material is selected from the group consisting of oil-based foundations, emollient-based foundations, alcohol-based foundation, powder-based foundations, mineral-based foundations, water-based foundations, and silicone-based foundations.

8. A process according to claim 2 wherein the cosmetic application tool is selected from the group consisting of sponges, brushes and air-brushes.

9. A process for application of a liquid cosmetic material to human skin, the process comprising:
a) providing at least one cosmetic material;
b) providing a silicone surface;
c) providing a cosmetic application tool;
d) applying the cosmetic material to the cosmetic application tool;
e) rubbing the cosmetic application tool bearing the at least one cosmetic material against the silicone surface to generate friction heat to increase the temperature of the cosmetic material; and
f) subsequently applying the heated cosmetic material to human skin.

10. A process according claim 9 wherein the at least one cosmetic material is a foundation material.

11. A process according to claim 10 wherein the foundation material is selected from the group consisting of oil-based foundations, emollient-based foundations, alcohol-based foundation, powder-based foundations, mineral-based foundations, water-based foundations, and silicone-based foundations.

12. A process according to claim 9 wherein the cosmetic application tool is selected from the group consisting of sponges, brushes and air-brushes.

13. A process according to claim 9 wherein the at least one cosmetic material comprises at least two cosmetic materials, and further comprising mixing the at least two cosmetic materials together with the cosmetic application tool.

14. A process according to claim 13, wherein the at least two cosmetic materials include at least one foundation material and at least one additive.

15. A process according to claim 14 wherein the at least one additive comprises a translucent powder material.

16. A storage container for a cosmetic material having an open position and a closed position, the storage container comprising:
a) a first section having a compartment formed therein for containing the cosmetic material, the first section having a first sealing surface, and
b) a second section having a silicone surface for frictionally heating the cosmetic material, the second section having a second sealing surface, wherein the first sealing surface and the second sealing surface cooperate to seal the container in the closed position.

17. A storage container according to claim 16 further comprising a hinge for connecting the first section and the second section.

18. A storage container according to claim 16 further comprising a lock for securing the storage container in the closed position.

19. A storage container according to claim 16 wherein the first section and the second section are cylindrical.

20. A storage container according to claim 16 wherein the first section and the second section have a hexagonal cross-section.
